(19) United States
(12) Patent Application Publication
(10) Pub. No.: US 2011/0245311 A1
(43) Pub. Date: Oct. 6, 2011

(54) Histidine Derivatives

(76) Inventor: Dennis Wright, Germantown Hills, IL (US)

(21) Appl. No.: 12/918,995

(22) PCT Filed: Apr. 2, 2010
(86) PCT No.: PCT/US2010/029818

§ 371 Date: Aug. 23, 2010

(51) Int. Cl. A61K 31/417; C07D 233/64; C12N 5/09; C12N 1/20

(52) U.S. Cl. 514/400; 548/334.1; 435/375; 435/252.1

(57) ABSTRACT

Disclosed herein are the compositions and methods for a compound of Formula Ia or Ib.

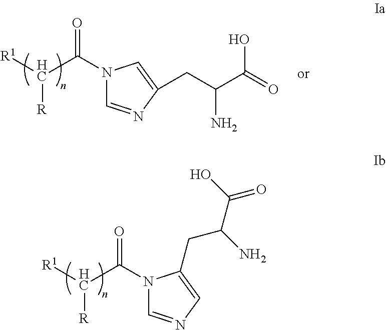

### CROSS-REFERENCE TO RELATED APPLICATIONS

This application claims the benefit under 35 U.S.C. §119 (e) of U.S. provisional application Ser. No. 61/211,799, filed Apr. 3, 2009. The content of this application is hereby incorporated by reference into the present disclosure.

### BACKGROUND OF THE INVENTION

In 2009, cancer is expected to cause about 560,000 death in the United States, and is responsible for about 25% of all deaths. Newly diagnosed cancer cases will be about 1.5 million in the United States alone in 2009. The most commonly occurring cancer in men is prostate cancer (about 25% of new cases) and in women is breast cancer (also about 25%). In the developed world, one in three people will develop cancer during their lifetimes. If all cancer patients survived and cancer occurred randomly, the lifetime odds of developing an second primary cancer would be one in nine. However, cancer survivors have an increased risk of developing a second primary cancer.

Currently, cancer sufferers who are non-responsive to chemotherapy and radiation therapy have limited therapeutic options. Aggressive treatments are typically associated with severe side effects. There is a need to develop new cancer therapies, especially those that cause fewer or less severe side effects.

Bacteria are the cause of another category of diseases. Gram-negative bacteria are those bacteria that do not retain crystal violet dye in the Gram staining protocol. The pathogenic capability of Gram-negative bacteria is often associated with certain components of Gram-negative cell walls, in particular the lipopolysaccharide (also known as LPS or endotoxin) layer. In humans, LPS triggers an innate immune response characterized by cytokine production and immune system activation. Inflammation is a common result of cytokine (from the Greek cyto, cell and kinesis, movement) production, which can also produce host toxicity.

An antibiotic is a substance or compound that kills, or inhibits the growth of, bacteria. Most currently ongoing research projects target Gram-positive bacteria, let alone that a large number of patients such as those from developing countries suffer from Gram-negative bacteria infections. Therefore, there is a strong need to develop new antibiotics especially those that target Gram-negative bacteria.

### SUMMARY OF THE INVENTION

In one aspect of the invention, there is provided a compound of Formula Ia or Ib:

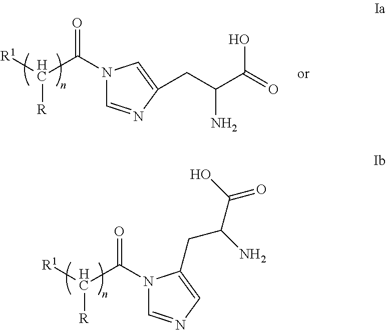

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer, a pharmaceutically acceptable salt or a prodrug thereof.
   In one aspect, there is provided a method of preparing a compound of Formula Ia or Ib:

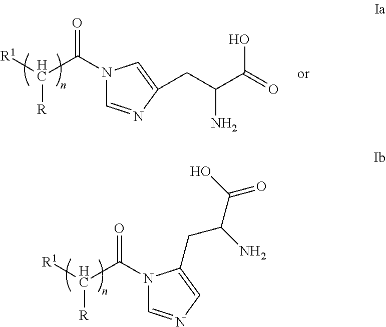

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer thereof,
comprising treating a compound of formula III:

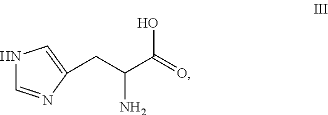

or a stereoisomer thereof,
with an acyl halide of formula IV:

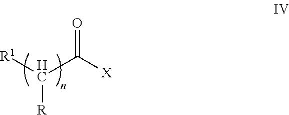

wherein
X is halo;
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
   under suitable reaction conditions to give the compound of formula Ia or Ib, respectively.

In one aspect, there is provided a method for treating a tumor in a subject, comprising administering to the subject an effective amount of a compound of Formula Ia or Ib:

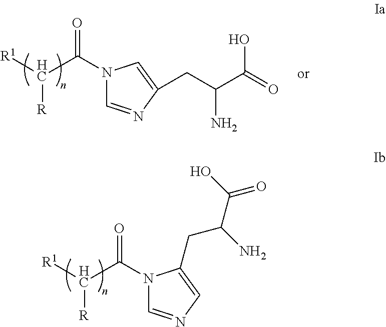

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer, a pharmaceutically acceptable salt or a prodrug thereof, thereby treating the tumor in the subject.

In one aspect, there is provided a method for treating microbial infection in a subject, comprising administering to the subject an effective amount of a compound of Formula Ia or Ib:

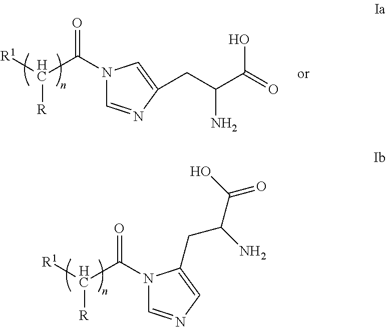

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer, a pharmaceutically acceptable salt or a prodrug thereof, thereby treating the microbial infection in the subject.

### DETAILED DESCRIPTION OF THE INVENTION

Before the present compositions and methods are described, it is to be understood that the invention is not limited to the particular compounds, compositions, methodologies, protocols, cell lines, assays, and reagents described, as these may vary. It is also to be understood that the terminology used herein is intended to describe particular embodiments of the present invention, and is in no way intended to limit the scope of the present invention as set forth in the appended claims.

### 1. DEFINITIONS

It must be noted that as used herein, and in the appended claims, the singular forms “a,” “an,” and “the” include plural references unless the context clearly dictates otherwise.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods, devices, and materials are now described. All publications cited herein are incorporated herein by reference in their entirety for the purpose of describing and disclosing the methodologies, reagents, and tools reported in the publications that might be used in connection with the invention. Nothing herein is to be construed as an admission that the invention is not entitled to antedate such disclosure by virtue of prior invention.

The practice of the present invention will employ, unless otherwise indicated, conventional methods of chemistry, biochemistry, molecular biology, cell biology, genetics, immunology, and pharmacology, within the skill of the art. Such techniques are explained fully in the literature. (See, e.g., Gennaro, A. R., ed. (1990) Remington's Pharmaceutical Sciences, 18^{th} ed., Mack Publishing Co.; Colowick, S. et al., eds., Methods In Enzymology, Academic Press, Inc.; D. M. Weir, and C. C. Blackwell, eds. (1986) Handbook of Experimental Immunology, Vols. I-IV, Blackwell Scientific Publications; Maniatis, T. et al., eds. (1989) Molecular Cloning: A Laboratory Manual, 2^{nd} edition, Vols. I-III, Cold Spring Harbor Laboratory Press; Ausubel, F. M. et al., eds. (1999) Short Protocols in Molecular Biology, 4^{th} edition, John Wiley & Sons; Ream et al., eds. (1998) Molecular Biology Techniques: An Intensive Laboratory Course, Academic Press; Newton & Graham eds. (1997) PCR (Introduction to Biotechniques Series), 2nd ed., Springer Verlag).

The term “alkyl” refers to saturated monovalent hydrocarbyl groups having from 1 to 4 carbon atoms, or alternatively from 1 to 2 carbon atoms, or alternatively from 1 to 3 carbon atoms, or alternatively 1, 2, 3, or 4 carbon atoms. This term is exemplified by groups such as methyl, ethyl, n-propyl, iso-propyl, n-butyl, t-butyl, and the like.

The term “alkoxy” refers to the group alkyl-O— that includes, by way of example, methoxy, ethoxy, and the like.

The term “aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is the aryl group. Preferred aryls include phenyl and naphthyl.

The term “substituted aryl” refers to aryl groups, as defined herein, which are substituted with from 1 to 4, particularly 1 to 3, substituents selected from the group consisting of hydroxy, alkyl, halo, alkoxy, carboxyl, and cyano.

The term “carboxyl” refers to —COOH or salts thereof.

“Comprising” is intended to mean that the compositions and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude trace contaminants from the isolation and purification method and pharmaceutically acceptable carriers, such as phosphate buffered saline, preservatives and the like. “Consisting of” shall mean excluding more than trace elements of other ingredients and substantial method steps for administering the compositions of this invention or process steps to produce a composition or achieve an intended result. Embodiments defined by each of these transition terms are within the scope of this invention.

The term “cyano” refers to the group —CN.

The term “halo” or “halogen” refers to fluoro, chloro, bromo, and iodo.

The term “hydroxy” or “hydroxyl” refers to the group —OH.

The term “heteroaryl” refers to an aromatic ring of from 1 to 15 carbon atoms, preferably from 1 to 10 carbon atoms, and 1 to 4 heteroatoms within the ring selected from the group consisting of oxygen, nitrogen, and sulfur. Such heteroaryl groups can have a single ring (e.g., pyridinyl, furyl, or thienyl) or multiple condensed rings (e.g., indolizinyl or benzothienyl) provided the point of attachment is through a ring containing the heteroatom and that ring is aromatic. The nitrogen and/or sulfur ring atoms can optionally be oxidized to provide for the N-oxide or the sulfoxide, and sulfone derivatives. Examples of heteroaryls include but are not limited to, pyridinyl, pyrrolyl, indolyl, thiophenyl, thienyl, and furyl.

The term “substituted heteroaryl” refers to heteroaryl groups that are substituted with from 1 to 3 substituents selected from the same group of substituents defined for substituted aryl.

A “subject” of diagnosis or treatment is a mammal, including a human. Non-human mammals subject to diagnosis or treatment include, for example, murine, such as rats, mice, canine, such as dogs, leporids, such as rabbits, simians, livestock, such as bovines or porcines, sport animals such as equines, and pets.

The term “pharmaceutically acceptable salt” refers to pharmaceutically acceptable salts of a compound, which salts are derived from a variety of organic and inorganic counter ions well known in the art, and include, by way of example only, sodium, potassium, calcium, magnesium, ammonium, tetraalkylammonium, and the like; and, when the molecule contains a basic functionality, salts of organic or inorganic acids, such as hydrochloride, hydrobromide, tartrate, mesylate, acetate, maleate, oxalate, and the like.

The terms “stereoisomer” or “stereoisomers” refer to compounds that differ in the chirality of one or more stereocenters and/or the configuration of a double bond (E/Z isomers). Stereoisomers include enantiomers and diastereomers.

The term “prodrug” as used herein, refers to compounds of the invention that include chemical groups which, in vivo, can be converted into the carboxylic acid group or an amino group. Prodrugs of a compound are prepared by modifying functional groups present in the compounds in such a way that the bonds are cleaved, either in routine manipulation or in vivo, to the parent compounds. Prodrugs include, but are not limited to, compounds wherein hydroxyl or amine groups are bonded to any group that, when administered to a subject, cleave to form a free hydroxyl or amino, group, respectively. Examples of prodrugs include, but are not limited to, acetyl and benzoyl derivatives of amine functional groups in the compounds of the invention and the like and for the carboxylic acid moiety, a prodrug selected from, e.g., esters including, but not limited to, those derived from alkyl alcohols and amides, particularly amides derived from amines.

A “carrier” refers to any diluents, excipients, or carriers that may be used in the compositions of the invention. A “pharmaceutically acceptable carrier” refers to a carrier that is acceptable for any pharmaceutical use. Pharmaceutically acceptable carriers include ion exchangers, alumina, aluminum stearate, lecithin, serum proteins, such as human serum albumin, buffer substances, such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Suitable pharmaceutical carriers are described in Remington's Pharmaceutical Sciences, Mack Publishing Company, a standard reference text in this field. They are preferably selected with respect to the intended form of administration, that is, oral tablets, capsules, elixirs, syrups and the like, and consistent with conventional pharmaceutical practices.

The term “excipient” as used herein means an inert or inactive substance used in the production of pharmaceutical products or other tablets, including without limitation any substance used as a binder, disintegrant, coating, compression/encapsulation aid, cream or lotion, lubricant, parenteral, sweetener or flavoring, suspending/gelling agent, or wet granulation agent. Binders include, e.g., carbopol, povidone, xanthan gum, etc.; coatings include, e.g., cellulose acetate phthalate, ethylcellulose, gellan gum, maltodextrin, etc.; compression/encapsulation aids include, e.g., calcium carbonate, dextrose, fructose dc, honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams and lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; parenterals include, e.g., mannitol, povidone, etc.; plasticizers include, e.g., dibutyl sebacate, polyvinylacetate phthalate, etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

“An effective amount” refers to the amount of a compound or composition of the invention to induce a desired biological and/or therapeutic result. That result can be alleviation of the signs, symptoms, or causes of a disease, or any other desired alteration of a biological system. The effective amount will vary depending upon the specific compound or composition used, the dosing regimen, timing of administration, the subject and disease condition being treated, the weight and age of the subject, the severity of the disease condition, the manner of administration and the like, all of which can be determined readily by one of ordinary skill in the art.

It is understood that the above definitions are not intended to include impermissible substitution patterns (e.g., monosubstituted aryl substituted with 6 halo groups or methyl substituted with 5 fluoro groups). Such impermissible substitution patterns are well known to the skilled artisan.

A “treating,” “treatment” and the like refer to obtaining a desired pharmacologic and/or physiologic effect. The effect can be prophylactic in terms of completely or partially preventing a disease or disorder or sign or symptom thereof, and/or can be therapeutic in terms of a partial or complete cure for a disorder and/or adverse effect attributable to the disorder. Examples of “treatment” include but are not limited to: preventing a disease from occurring in a subject that may be predisposed or at risk of a disease, such as infection or tumor, but has not yet been diagnosed as having it; inhibiting a disease, i.e., arresting its development; and/or relieving or ameliorating the symptoms of disease or reducing the likelihood of recurrence of the disease, such as infection or tumor. As is understood by those skilled in the art, “treatment” can include systemic amelioration of the symptoms associated with the pathology and/or a delay in onset of symptoms.

### 2. COMPOUNDS AND COMPOSITIONS OF THE INVENTION

In one aspect of the invention, there is provided a compound of Formula Ia or Ib:

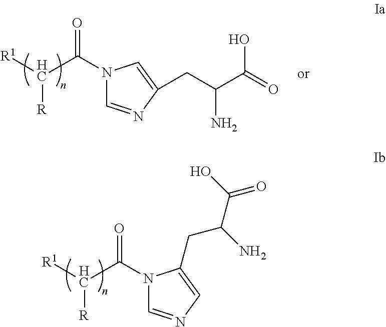

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer, a pharmaceutically acceptable salt or a prodrug thereof.

In some embodiments, a pharmaceutically acceptable salt of formula Ia or Ib can be prepared by modifying the structures identified above by treatment with bases such as, but not limited to, sodium hydroxide, potassium hydroxide, calcium carbonate, magnesium carbonate, etc. to make salt with counter ions such as, sodium, potassium, calcium, magnesium, ammonium, tetraalkylammonium etc. In some embodiments, an example of a pharmaceutically acceptable salt is a sodium salt or a potassium salt.

In some embodiments, a prodrug of formula Ia or Ib can be prepared by preparing esters of the compounds of formula Ia or Ib by treatment with alcohols such as, but not limited to, methanol, ethanol, propanol, i-propanol, etc. to make esters. In some embodiments, an example of a prodrug is a methyl ester or ethyl ester.

In some embodiments, n is 0. In some embodiments, n is 1. In some embodiments, n is 2 or 3. In some embodiments, n is 2. In some embodiments, n is 3.

In some embodiments, R is hydrogen.

In some embodiments, R is independently hydrogen or halo.

In some embodiments, R is independently hydrogen or C₁-C₃ alkyl.

In some embodiments, R is independently hydrogen or methyl.

In some embodiments, R¹ is aryl or substituted aryl.

In some embodiments, R¹ is aryl.

In some embodiments, R¹ is phenyl.

In some embodiments, R¹ is aryl substituted with halo or methyl.

In some embodiments, n is 0 or 1; R is hydrogen or methyl; and R¹ is aryl or substituted aryl.

In some embodiments, n is 1; R is hydrogen; and R¹ is substituted or unsubstituted aryl having a 5-7 membered ring.

In some embodiments, n is 1; R is hydrogen; and R¹ is unsubstituted aryl having a 5-7 membered ring.

In some embodiments, a compound is selected from the group consisting of:
2-amino-3-(1-(2-phenylacetyl)-1H-imidazol-4-yl)propanoic acid;
(S)-2-amino-3-(1-(2-phenylacetyl)-1H-imidazol-4-yl)propanoic acid;
(R)-2-amino-3-(1-(2-phenylacetyl)-1H-imidazol-4-yl)propanoic acid;
2-amino-3-(1-(2-phenylacetyl)-1H-imidazol-5-yl)propanoic acid;
(S)-2-amino-3-(1-(2-phenylacetyl)-1H-imidazol-5-yl)propanoic acid;
(R)-2-amino-3-(1-(2-phenylacetyl)-1H-imidazol-5-yl)propanoic acid;

or a pharmaceutically acceptable salt or a prodrug thereof.

In some embodiments, a compound is selected from IIa or IIb:

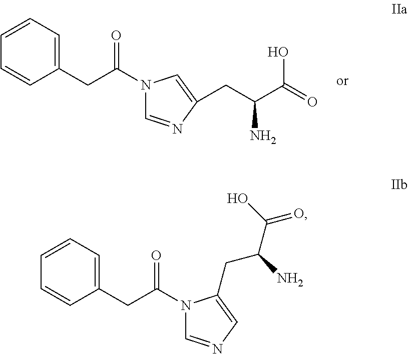

or a pharmaceutically acceptably salt or a prodrug thereof.

All possible tautomers of the compounds of the invention are within the scope of the invention.

In some embodiments, compositions are provided, comprising a compound of any of the above embodiment and a carrier. In one aspect, the carrier is a pharmaceutically acceptable carrier.

### 3. METHODS OF PREPARATION

Applicants have unexpectedly found that the methods of preparation of the present invention result in the N-acylation of the imidazole ring, which is substituted with the amino acid, without protecting the amino group with a protecting group such as a benzoyl, or t-Boc (t-butyl carbamate), etc. Under suitable reaction conditions, the compounds of the invention are prepared in near quantitative yield.

In some embodiments, it is contemplated that the methods of preparation of the present invention can be used to synthesize any N-acylated ring that is substituted with an amino acid without the protection of the amino group. In some embodiments, it is contemplated that the methods of preparation of the present invention can be used to synthesize an N-acylated aryl, heteroaryl, or a heterocyclic ring that is substituted with an amino acid, without the protection of the amino group.

Without being limited by any theory, it is contemplated that the acyl halide does not react at the primary amine of an alpha amino acid, such as, but not limited to, histidine or tryptophan etc., when a secondary amine on the ring such as, but not limited to, heteroaryl (eg. imidazole, indole, etc.) is available on the alpha amino acid.

In one aspect, there is provided a method of preparing a compound of Formula Ia or Ib:

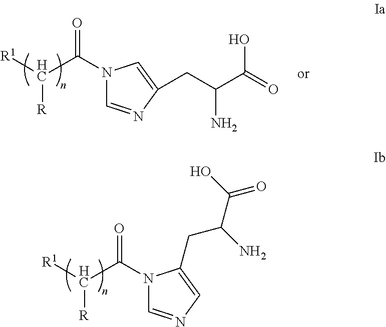

wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer thereof,
comprising treating a compound of formula III:

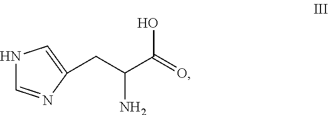

or a stereoisomer thereof,
with an acyl halide of formula IV:

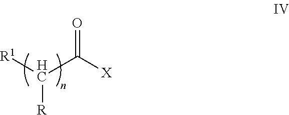

wherein
X is halo;
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
   under suitable reaction conditions to give the compound of formula Ia or Ib, respectively.

The above noted embodiments of the compounds of the invention are also embodiments for the above noted aspect of the method of preparation.

In some embodiments, the suitable reaction conditions in the above noted aspect comprise carrying out the reaction below room temperature. In some embodiments, the suitable reaction conditions in the above noted aspect comprise carrying out the reaction below −5° C. In some embodiments, the suitable reaction conditions for the preparation of compound of formula Ib comprise carrying out the reaction below room temperature.

In some embodiments, the suitable reaction conditions in the above noted aspect comprise carrying out the reaction at or above room temperature. In some embodiments, the suitable reaction conditions in the above noted aspect comprise carrying out the reaction at or above 50° C. In some embodiments, the suitable reaction conditions for the preparation of compound of formula Ia comprise carrying out the reaction at or above room temperature.

In some embodiments, X is chloro, fluoro, or bromo; n is 1; R is hydrogen or methyl; and R¹ is aryl or substituted aryl.

In some embodiments, X is chloro; n is 1; R is hydrogen; and R¹ is aryl or substituted aryl.

In some embodiments, X is chloro; n is 1; R is hydrogen; and R¹ is aryl.

### 4. METHODS OF USE

The compounds and compositions of the invention can be suitable for treating cancer or a bacterial infection. The histidine derivatives of any of the above embodiments, in particular derivative2-amino-3-(3-phenylacetylimidazol-4-yl)propanoic acid, have been observed to have potent antineoplastic and antibiotic activities. These activities, at least in part, may be due to the compounds' ability to be incorporated, in place of histidine, into newly synthesized polypeptides in a cell, especially in fast-growing cells such as cancer cells or bacteria. Polypeptides or proteins containing these histidine derivatives are then recognized as deleterious to the cells resulting in activation of the apoptosis pathway leading to death of the cells.

The antineoplastic activity of the compounds have now been shown with U-87, a human glioblastoma/astrocytoma cell line and HepG2, a human liver carcinoma cell line with no observed toxicity. Prostate cancer cells, breast cancer cells, melanoma cancer cells, lymphoma cancer cells can further be tested and are expected to exhibit antineoplastic activity for the compounds of the invention. The anticipated antineoplastic activity of the compounds of the invention is based on the demonstrated antineoplastic activity in the glioblastoma/astrocytoma cell line and liver carcinoma cell line. Further, the compounds of the invention have herein been observed to activate the apoptosis pathway in a cell, in particular a proliferating or fast-growing cell in which nascent peptide synthesis is active. Inhibiting cancer cell growth or promoting cell death by activating the apoptosis pathway is known in the art as a universal approach to treat cancer. See, e.g., Gerl and Vaux (2005) Carcinogenesis 26(2):263-70. Further, it has been shown that antineoplaston A10 (3-phenylacetyl-2,6-piperidinodione), an amino acid derivative, can be used to treat multiple types of cancers, such as brain tumor, hepatocellular carcinoma, breast cancer, colorectal cancer, through induction of apoptosis (Qu et al. (2007) Anticancer Res. 27(4B):2427-31). Therefore, by activating the apoptosis pathway in a cancer cell leading to the cell death, the compounds of the invention can be used to treat cancer of any type or origin, such as but not limited to adenocarcinoma, leukemia, lymphoma, melanoma, myeloma, sarcoma or teratocarcinoma, or the cancer of adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus.

Further, the histidine derivatives of the invention are capable of crossing the blood brain barrier (BBB), making these compounds particularly useful in treating tumors or infections in the brain or the spinal cord. It is known in the art that neutral L-amino acids have various rates of movement into the brain. Phenylalanine, leucine, tyrosine, isoleucine, valine, tryptophan, methionine, histidine and L-dihydroxy-phenylalanine (l-DOPA) may enter as rapidly as glucose. These essential amino acids cannot be synthesized by the brain and, therefore, must be supplied from protein breakdown and diet. Alternatively, various pharmaceutically acceptable carriers, such as a nanoparticle as disclosed in Schroder and Sabel (1996) Brain Research 710(1-2):121-124, or a blood brain barrier permeation peptide as disclosed in United States Patent Application Publication No.: 20060039859.

Accordingly, in one aspect, the invention provides a method for treating a tumor, in a subject, comprising, or alternatively consisting essentially of, or alternatively consisting of, administering to the subject an effective amount of a compound of any of the above embodiments, thereby treating the tumor in the subject.

In another aspect, provided is a method for inhibiting the growth of a tumor cell in a subject, comprising, or alternatively consisting essentially of, or alternatively consisting of, contacting the tumor cell with an effective amount of a compound of any of the above embodiments. The contacting can be in vitro or in vivo. When conducted in vitro, the method provides a screen to assay for new pharmaceutical agents or combination therapies to treat infections in vivo. When conducted in vivo in an animal model such as a nude mouse model, it provides a pre-clinical screen for safety and efficacy for therapies comprising the administration of the compound of this invention.

Tumors suitable for the treatment by the compounds, composition and method of the invention included, without limitation, adenocarcinoma, leukemia, lymphoma, melanoma, myeloma, sarcoma or teratocarcinoma. Further, without limitation, examples of tumors suitable for the treatment include a tumor of one or more of adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus.

In some embodiments, the tumor is a gastrointestinal tumor. Non-limiting examples of gastrointestinal tumors include gastrointestinal stromal tumors (GIST), esophageal cancer, stomach cancer, liver cancer, gallbladder cancer, pancreatic cancer, colorectal cancer or anal cancer. In some embodiments, the tumor is a brain tumor, such as but not limited to, glioblastoma, glioblastoma multiforme, medulloblastoma, astrocytoma, pilocytic astrocytoma, CNS lymphoma, brainstem glioma, germinoma, meningioma, oligodendroglioma, schwannoma, craniopharyngioma, ependymoma, mixed gliomas, or brain metastasis.

The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The compounds and compositions described herein and the pharmaceutically acceptable salts thereof are administered via oral, nasal, parenteral, subcutaneous, intramuscular, intravenous or topical routes. Generally, it is contemplated that the compounds and compositions are to be administered in dosages ranging from about 0.10 milligrams (mg) up to about 1000 mg per day, although variations will necessarily occur, depending, as noted above, on the target tissue, the subject, and the route of administration. In preferred embodiments, the compounds or compositions are administered orally, once or twice a day.

Also provided is a method for treating microbial infection in a subject, comprising, or alternatively consisting essentially of, or alternatively consisting of, administering to the subject an effective amount of a compound of any of the above embodiments, thereby treating the microbial infection in the subject.

Further provided, in another aspect, is a method for inhibiting the growth of a microorganism, comprising, or alternatively consisting essentially of, or alternatively consisting of, contacting the microorganism with an effective amount of a compound of any of the above embodiments. The contacting is conducted in vitro or in vivo. When conducted in vitro, the method provides a screen to assay for new pharmaceutical agents or combination therapies to treat infections in vivo. When conducted in vivo in an animal model such as a guinea pig infected with bacteria, it provides a pre-clinical screen for safety and efficacy for therapies comprising the administration of the compound of this invention.

In one aspect, the microbial infection is bacterial infection or the microorganism is a bacterium. In one aspect, the bacterium is Gram-negative bacterium, such as but not limited to, Proteus vulgaris, Klebsiella pneumoniae, Escherichia coli, or Pseudomomas aeruginosa.

Yet further provided is a method for activating apoptosis in a cell, comprising, or alternatively consisting essentially of, or alternatively consisting of, contacting the cell with an effective amount of a compound of any of the above embodiments. The contacting is conducted in vitro or in vivo.

Still further provided is a method for activating caspase 3 in a cell, comprising, or alternatively consisting essentially of, or alternatively consisting of, contacting the cell with an effective amount of a compound of any of the above embodiments. The contacting is conducted in vitro or in vivo.

As used herein, “activating apoptosis” intends increasing the expression or biological activity of any component of the apoptosis pathway in a cell that positively regulates apoptosis, such as but not limited to caspase 3, caspase 6, caspase 7, caspase 14, caspase 2, caspase 8, caspase 9, caspase 10, or caspase 14. Activation of apoptosis can be detected with methods known in the art, some of which are reviewed in Sgnoc and Gruber (1998) Exp Gerontol. 33(6):525-33, the content of which is incorporated by reference into the current disclosure in its entirety. Commercial kits are also available for detection of apoptosis, such as the cytokeratin-based apoptosis detection kit, the DNA-based apoptosis detection kit, or the mitochondria-based apoptosis detection kit, from vendors such as Enzo Life Sciences (Farmingdale, N.Y.).

As used herein, “activating caspase 3” refers to increasing the mRNA expression or protein expression of the caspase 3 gene, increasing the biological or enzymatic or protease activity of the caspase 3 protein, or inhibiting genes, proteins, RNA, or small molecules that inhibit the expression or activity of the caspase 3 gene or protein. Methods of measuring the expression or activity of caspase 3, such as PCR and protease assays, are known in the art.

The cells can be normal cells, tumor cells, primary cells, cells of cell lines, or stem cells.

In one aspect, the cell is a tumor cell, such as but not limited to a cell of adenocarcinoma, leukemia, lymphoma, melanoma, myeloma, sarcoma or teratocarcinoma. Further, without limitation, examples of tumor cells include a tumor cell of one or more of adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus.

A subject as used herein or the origin of cell herein, can be a vertebrate, preferably a mammal, more preferably a human. Mammals include, but are not limited to, murines, rats, rabbit, simians, bovines, ovine, porcine, canines, feline, farm animals, sport animals, pets, equine, and primate, particularly human. Besides being useful for human treatment, the present invention is also useful for veterinary treatment of companion mammals, exotic animals and domesticated animals, including mammals, rodents, and the like.

The compounds and compositions of this invention are useful for the manufacture of medicaments for treating any of the tumors or microbial infections as disclosed above. Also provided are compounds or compositions of any of the above embodiments for use to treat any of the tumors or microbial infections as disclosed above.

### 5. COMPOUND PREPARATION

The compounds of this invention can be prepared from readily available starting materials using, for example, the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts (1999) Protecting Groups in Organic Synthesis, 3rd Edition, Wiley, New York, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents, and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989).

### Synthesis of Compounds of the Invention

The compounds of this invention are prepared by, for example, the synthetic protocols illustrated in Scheme A.

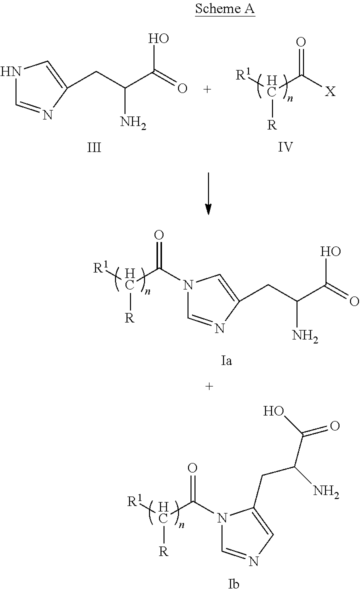

In Scheme A, the substituents n, R, R¹, and X are as defined herein. Histidine III and a suitable acyl halides IV are commercially available. Histidine III and acyl halide IV are reacted in the presence of a suitable solvent. Typically, the acyl halide itself acts as a solvent. The reaction is carried out below room temperature or below −5° C. to result in a compound of formula Ib. Alternatively, the reaction can be carried our at or above room temperature to result in a compound of formula Ia. Upon reaction completion, the intermediates are treated with a base to remove the acid from the reaction mixture. Such bases are well known in the art and include, without limitation, sodium hydroxide, potassium hydroxide, sodium bicarbonate etc. The product(s) can be recovered by conventional techniques such as neutralization, extraction, precipitation, chromatography, filtration, and the like.

### 6. PHARMACEUTICAL FORMULATIONS AND ROUTES OF ADMINISTRATION

The compositions of the present invention can be delivered directly or in pharmaceutical compositions along with suitable carriers or excipients, as is well known in the art. Present methods of treatment can comprise administration of an effective amount of a compound of the invention to a subject in need. In a preferred embodiment, the subject is a mammalian subject, and in a most preferred embodiment, the subject is a human subject.

An effective amount of such agents can readily be determined by routine experimentation, as can the most effective and convenient route of administration, and the most appropriate formulation. Various formulations and drug delivery systems are available in the art. See, e.g., Gennaro, A. R., ed. (1995) Remington's Pharmaceutical Sciences, supra.

Suitable routes of administration may, for example, include oral, rectal, topical, nasal, pulmonary, ocular, intestinal, and parenteral administration. Primary routes for parenteral administration include intravenous, intramuscular, and subcutaneous administration. Secondary routes of administration include intraperitoneal, intra-arterial, intra-articular, intracardiac, intracisternal, intradermal, intralesional, intraocular, intrapleural, intrathecal, intrauterine, and intraventricular administration. The indication to be treated, along with the physical, chemical, and biological properties of the drug, dictate the type of formulation and the route of administration to be used, as well as whether local or systemic delivery would be preferred.

Pharmaceutical dosage forms of a compound of the invention may be provided in an instant release, controlled release, sustained release, or target drug-delivery system. Commonly used dosage forms include, for example, solutions and suspensions, (micro-) emulsions, ointments, gels and patches, liposomes, tablets, dragees, soft or hard shell capsules, suppositories, ovules, implants, amorphous or crystalline powders, aerosols, and lyophilized formulations. Depending on route of administration used, special devices may be required for application or administration of the drug, such as, for example, syringes and needles, inhalers, pumps, injection pens, applicators, or special flasks. Pharmaceutical dosage forms are often composed of the drug, an excipient(s), and a container/closure system. One or multiple excipients, also referred to as inactive ingredients, can be added to a compound of the invention to improve or facilitate manufacturing, stability, administration, and safety of the drug, and can provide a means to achieve a desired drug release profile. Therefore, the type of excipient(s) to be added to the drug can depend on various factors, such as, for example, the physical and chemical properties of the drug, the route of administration, and the manufacturing procedure. Pharmaceutically acceptable excipients are available in the art and include those listed in various pharmacopoeias. (See, e.g., the U.S. Pharmacopeia (USP), Japanese Pharmacopoeia (JP), European Pharmacopoeia (EP), and British pharmacopeia (BP); the U.S. Food and Drug Administration (www.fda.gov) Center for Drug Evaluation and Research (CEDR) publications, e.g., Inactive Ingredient Guide (1996); Ash and Ash, Eds. (2002) Handbook of Pharmaceutical Additives, Synapse Information Resources, Inc., Endicott N.Y.; etc.)

Pharmaceutical dosage forms of a compound of the present invention may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present invention can include one or more physiologically acceptable inactive ingredients that facilitate processing of active molecules into preparations for pharmaceutical use.

Proper formulation is dependent upon the desired route of administration. For intravenous injection, for example, the composition may be formulated in aqueous solution, if necessary using physiologically compatible buffers, including, for example, phosphate, histidine, or citrate for adjustment of the formulation pH, and a tonicity agent, such as, for example, sodium chloride or dextrose. For transmucosal or nasal administration, semisolid, liquid formulations, or patches may be preferred, possibly containing penetration enhancers. Such penetrants are generally known in the art. For oral administration, the compounds can be formulated in liquid or solid dosage forms, and as instant or controlled/sustained release formulations. Suitable dosage forms for oral ingestion by a subject include tablets, pills, dragees, hard and soft shell capsules, liquids, gels, syrups, slurries, suspensions, and emulsions. The compounds may also be formulated in rectal compositions, such as suppositories or retention enemas, e.g., containing conventional suppository bases such as cocoa butter or other glycerides.

Solid oral dosage forms can be obtained using excipients, which may include fillers, disintegrants, binders (dry and wet), dissolution retardants, lubricants, glidants, antiadherants, cationic exchange resins, wetting agents, antioxidants, preservatives, coloring, and flavoring agents. These excipients can be of synthetic or natural source. Examples of such excipients include cellulose derivatives, citric acid, dicalcium phosphate, gelatine, magnesium carbonate, magnesium/sodium lauryl sulfate, mannitol, polyethylene glycol, polyvinyl pyrrolidone, silicates, silicium dioxide, sodium benzoate, sorbitol, starches, stearic acid or a salt thereof, sugars (i.e. dextrose, sucrose, lactose, etc.), talc, tragacanth mucilage, vegetable oils (hydrogenated), and waxes. Ethanol and water may serve as granulation aides. In certain instances, coating of tablets with, for example, a taste-masking film, a stomach acid resistant film, or a release-retarding film is desirable. Natural and synthetic polymers, in combination with colorants, sugars, and organic solvents or water, are often used to coat tablets, resulting in dragees. When a capsule is preferred over a tablet, the drug powder, suspension, or solution thereof can be delivered in a compatible hard or soft shell capsule.

In one embodiment, the compounds of the present invention can be administered topically, such as through a skin patch, a semi-solid, or a liquid formulation, for example a gel, a (micro-) emulsion, an ointment, a solution, a (nano/micro)-suspension, or a foam. The penetration of the drug into the skin and underlying tissues can be regulated, for example, using penetration enhancers; the appropriate choice and combination of lipophilic, hydrophilic, and amphiphilic excipients, including water, organic solvents, waxes, oils, synthetic and natural polymers, surfactants, emulsifiers; by pH adjustment; and use of complexing agents. Other techniques, such as iontophoresis, may be used to regulate skin penetration of a compound of the invention. Transdermal or topical administration would be preferred, for example, in situations in which local delivery with minimal systemic exposure is desired.

For administration by inhalation, or administration to the nose, the compounds for use according to the present invention are conveniently delivered in the form of a solution, suspension, emulsion, or semisolid aerosol from pressurized packs, or a nebuliser, usually with the use of a propellant, e.g., halogenated carbons derived from methane and ethane, carbon dioxide, or any other suitable gas. For topical aerosols, hydrocarbons like butane, isobutene, and pentane are useful. In the case of a pressurized aerosol, the appropriate dosage unit may be determined by providing a valve to deliver a metered amount. Capsules and cartridges of, for example, gelatin, for use in an inhaler or insufflator, may be formulated. These typically contain a powder mix of the compound and a suitable powder base such as lactose or starch.

Compositions formulated for parenteral administration by injection are usually sterile and can be presented in unit dosage forms, e.g., in ampoules, syringes, injection pens, or in multi-dose containers, the latter usually containing a preservative. The compositions may take such forms as suspensions, solutions, or emulsions in oily or aqueous vehicles, and may contain formulatory agents, such as buffers, tonicity agents, viscosity enhancing agents, surfactants, suspending and dispersing agents, antioxidants, biocompatible polymers, chelating agents, and preservatives. Depending on the injection site, the vehicle may contain water, a synthetic or vegetable oil, and/or organic co-solvents. In certain instances, such as with a lyophilized product or a concentrate, the parenteral formulation would be reconstituted or diluted prior to administration. Depot formulations, providing controlled or sustained release of a compound of the invention, may include injectable suspensions of nano/micro particles or nano/micro or non-micronized crystals. Polymers such as poly(lactic acid), poly(glycolic acid), or copolymers thereof, can serve as controlled/sustained release matrices, in addition to others well known in the art. Other depot delivery systems may be presented in form of implants and pumps requiring incision.

Suitable carriers for intravenous injection for the molecules of the invention are well-known in the art and include water-based solutions containing a base, such as, for example, sodium hydroxide, to form an ionized compound; sucrose or sodium chloride as a tonicity agent; and a buffer, for example, a buffer that contains phosphate. Co-solvents, such as, for example, polyethylene glycols, may be added. These water-based systems are effective at dissolving compounds of the invention and produce low toxicity upon systemic administration. The proportions of the components of a solution system may be varied considerably, without destroying solubility and toxicity characteristics. Furthermore, the identity of the components may be varied. For example, low-toxicity surfactants, such as polysorbates or poloxamers, may be used, as can polyethylene glycol or other co-solvents, biocompatible polymers such as polyvinyl pyrrolidone may be added, and other sugars and polyols may substitute for dextrose.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the invention, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is the therapeutic index, which can be expressed as the ratio LD₅₀/ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present compositions may, if desired, be presented in a pack or dispenser device containing one or more unit dosage forms containing the active ingredient. Such a pack or device may, for example, comprise metal or plastic foil, such as a blister pack; or glass and rubber stoppers such as in vials. The pack or dispenser device may be accompanied by instructions for administration. Compositions comprising a compound of the invention formulated in a compatible pharmaceutical carrier may also be prepared, placed in an appropriate container, and labeled for treatment of an indicated condition.

### 7. KITS AND ASSAYS

In one aspect, there is provided a kit comprising a compound of the invention, and optionally a pharmaceutically acceptable carrier.

The kits may further comprise suitable packaging and/or instructions for use of the compound of the invention. Kits may also comprise a means for the delivery of the compound of the invention, such as syringe, catheter, or other such devices. The kits may further comprise surgical tools.

The kits may also include other compounds for use in conjunction with the stent described herein. Such compounds include, but are not limited to, e.g., alcohol, analgesics, anesthetics, antiseptics, etc. These compounds can be provided in a separate form. The kits may include appropriate instructions for use of the compound of the invention, side effects, and any other relevant information. The instructions can be in any suitable format, including, but not limited to, e.g., printed matter, videotape, or computer readable disk.

In one embodiment, this invention provides a kit comprising a compound of the invention, optionally a pharmaceutically acceptable carrier, and instructions for use. In one embodiment, this invention provides a kit comprising a compound of the invention; packaging; and instructions for use.

These and other embodiments of the present invention will readily occur to those of ordinary skill in the art in view of the disclosure herein and are specifically contemplated.

Any of the compounds or compositions or kits of the invention can also be used to assay for new agents for inhibiting the growth of a tumor cell or a microorganism. In one embodiment, a candidate agent can be administered to a subject or be placed in contact with an appropriate test tumor cell or microorganism as compared to a compound, composition or kit of the invention. A suitable candidate is identified if the candidate agent shows better biological or therapeutic effect than the compound composition or kit of comparison.

### EXAMPLES

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art and from the foregoing description. Such modifications fall within the scope of the appended claims.

### Example 1

### Synthesis of (S)-2-amino-3-(3-phenylacetylimidazol-4-yl) propanoic acid (Compound A)

L-Histidine ((S)-2-amino-3-(1H-imidazol-4-yl) propanoic acid, 12.5 gram (g)) was added in a double beaker (e.g., a 150 milliliter (ml) beaker/w stir bar inside of a larger beaker (300 ml)) that was partially filled with crushed ice. An equivalent amount (12.5 ml) of phenylacetyl chloride was slowly added to the beaker while stirring. The temperature was not allowed to go over 50° C. The reactants were allowed to react for one hour while stirring until the completion of the reaction. The pH was checked at 15 minute intervals and was not allowed to go below 1.0 (1M NaOH was added when necessary). After completion, the pH of the reaction was adjusted to 7.0 by adding 1M NaOH (liquid). The product was purified by vacuum filtration (2× w/Whatman filter paper) and differential solubilities (H2O and CH3OH).

A thin layer chromatography (TLC); high performance liquid chromatography (HPLC); gas chromatography/mass spec (GC/MS); and infra-red (IR) spectroscopy of the resulting product were performed. The TLC confirmed the presence of a primary amine (ninhydrin detection) in the product. The HPLC data, after filtration/purification of the product, indicated single peak purity. The peak eluted earlier than the phenylacetic acid standard peak, indicating a single aromatic compound with higher molecular weight. GC/MS data was consistent with the predicted structure and molecular weight of the product. The IR indicated the presence of the OH bend as well as the carboxyl group. The melting point demonstrated a single compound which disintegrated at a specific temp.

### Example 2

### Antibiotic Activities

The antibiotic activity of S-2-amino-3(3-phenylacetylimidazol-4-yl)propanoic acid (Compound A) was tested with a panel of standard ATCC bacteria, including 13315 Proteus vulgaris, 13883 Klebsiella pneumoniae, 25922 Escherichia coli, 27853 and Pseudomomas aeruginosa.

Doubling dilutions of Compound A were tested on its antibiotic potentials. At concentrations of 2 mg/ml, 1 mg/ml, 500 mg/ml, 250 mg/ml, Compound A exhibited inhibitory effects. Plate counts were performed. All petri plates inoculated with 1 ml 0.5McFarland turbidity of corresponding ATCC strain. All control plates (i.e., Mueller-Hinton agar without Compound A were TNTC (too numerous to count, >300 CFU)—after overnight incubation at 37° C. Compound A did show inhibition of Gram negative strains at all concentrations tested. No inhibition was shown for Gram positives.

### Example 3

### Antineoplastic Activities

The antineoplastic activities of Compound A were tested with U-87, a human glioblastoma/astrocytoma cell line and HepG2, a human liver carcinoma cell line.

Cell viability with the U-87 cells (50,000/well) was tested in a microtiter plate (MTP) format in 100 μl Modified Eagles Medium (MEM), at pH 7.0, with or without Compound A. The cells were incubated at 37° C. for 72 hours followed by a cell count. The antineoplastic activity of Compound A was measured as a function of reduction of cell numbers and EC90 was determined as the concentration of Compound A resulting in greater than 90% decrease of cell numbers.

A dose response was observed with different concentrations of Compound A. Reproducibly the EC90 of Compound A was observed as 5 mM. The antineoplastic efficacy of Compound A is considerably higher that of phenylacetylglutamine, which has an EC90 of 50 mM.

The antineoplastic activity of Compound A was also measured with HepG2 cells. Similar to U-97, a dose response was observed with Compound A and the EC90 was 5 mM. Cell viability of a HepG2 cell line was performed on MTP (50 k cells/well—with 100 μl MEM supplement—including varying amounts of Compound A. A >90% decrease in cell viability was observed with a 5 mM concentration of Compound A.

Preclinical safety studies were performed with administration of different levels of doses of Compound A to mice. Toxicity was analyzed with standard clinical chemical and histopathological examination. Five mice (n=5) were treated with 300 mg/kg or 500 mg/kg orally (PO) with one hour pretreatment for PO. Autonomic signs/mortality was measured during the first hour and mortality was measured again at 3, 24, 48 and 72 hours after administration. No toxicity was observed.

### Example 4

### Antineoplastic Activities in Other Cancer Types

The antineoplastic activities of Compound A or any compound of the invention can be tested with other cell lines, including cells lines originated from, e.g., breast cancer cell lines, prostate cancer cell lines, melanoma cell lines and lymphoma cell lines.

Cell viability with the cell lines can be tested in a microtiter plate (MTP) format in 100 μl Modified Eagles Medium (MEM), at pH 7.0, with or without the compound being tested. The cells can be incubated at 37° C. for 72 hours followed by a cell count. The antineoplastic activity of the compound can be measured as a function of reduction of cell numbers and EC90 can be determined as the concentration of the compound. A decrease of cell number will indicate antineoplastic activity of the compound in the cell line.

A dose response can also observed with different concentrations of the compound being tested such as Compound A.

Preclinical safety studies can be performed with administration of different levels of doses of the compound being tested to mice. Toxicity can be analyzed with standard clinical chemical and histopathological examination. Five mice (n=5) can be treated with various doses of the compound orally (PO) with one hour pretreatment for PO. Autonomic signs/mortality can be measured during the first hour and mortality can be measured again at 3, 24, 48 and 72 hours after administration.

A prostate cancer cell line that can be tested with a compound of the invention is the LNCaP cell line, an immortalized, human, androgen-responsive prostate tumor cell line.

A breast cancer cell line that can be tested with a compound of the invention is the MCF-7, which was derived in the Michigan Cancer Foundation (from which it derives its name) in 1973.

A melanoma cell line that can be tested with a compound of the invention is the LND1 human melanoma cell line

A lymphoma cell line that can be tested with a compound of the invention is the FLK-1 cell line, which was established from bone marrow cells of a patient with follicular lymphoma.

### Example 5

### Activation of the Apoptotic Pathway

U-87 cells were used to test the effect of Compound A on the apoptotic pathway.

U-87 cells were seeded in MEM alone or in MEM supplemented with 5 mM of Compound A, in MTP. For in vitro caspase assay, the cell culture plates were removed from incubator, cells were lysed and centrifuged and the supernatant was tested for caspase levels (C3). Supernatants from each sample were tested side by side on a new microtitre plate by ELISA with AP labelled IgG C3 antibody, pNPP chromogen (Enzo Diagnostics, Farmingdale, N.Y.). Caspase assays were performed immediately after the addition of the MEM medium, with or without Compound A, and 18 hours after the addition.

Exponential increase, in the scale of several orders of magnitude, of caspase 3 was observed in U-87 cells treated with 5 mM Compound A. The increase was evidence because the 540 nm absorbance reading was maxed out and it was obvious visually.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

1. A compound of Formula Ia or Ib: 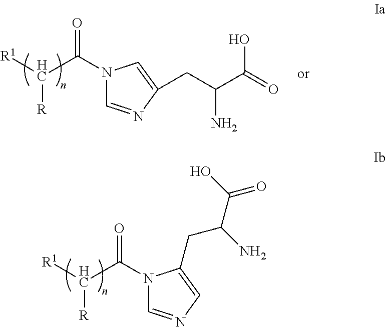
wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer, a pharmaceutically acceptable salt or a prodrug thereof.

2. (canceled)

3. The compound of claim 1, wherein n is 1.

4-5. (canceled)

6. The compound of claim 1, wherein R is independently hydrogen or halo.

7. The compound of claim 1, wherein R is independently hydrogen or C₁-C₃ substituted or unsubstituted alkyl.

8. The compound of claim 1, wherein R is independently hydrogen or methyl.

9. (canceled)

10. The compound of claim 1, wherein R¹ is unsubstituted aryl having a 5-7 membered ring.

11. The compound of claim 1, wherein R¹ is phenyl.

12. The compound of claim 1, wherein R¹ is aryl substituted with halo or methyl.

13. The compound of claim 1, wherein n is 0 or 1; R is hydrogen or methyl; and R¹ is aryl or substituted aryl.

14-15. (canceled)

16. The compound of claim 1, wherein the compound is selected from the group consisting of:
2-amino-3-(1-(2-phenylacetyl)-1H-imidazol-4-yl)propanoic acid;
(S)-2-amino-3-(1-(2-phenylacetyl)-1H-imidazol-4-yl)propanoic acid;
(R)-2-amino-3-(1-(2-phenylacetyl)-1H-imidazol-4-yl)propanoic acid;
2-amino-3-(1-(2-phenylacetyl)-1H-imidazol-5-yl)propanoic acid;
(S)-2-amino-3-(1-(2-phenylacetyl)-1H-imidazol-5-yl)propanoic acid; and
(R)-2-amino-3-(1-(2-phenylacetyl)-1H-imidazol-5-yl)propanoic acid;
or a pharmaceutically acceptable salt or a prodrug thereof.

17. The compound of claim 1, wherein the compound has Formula IIa or IIb: 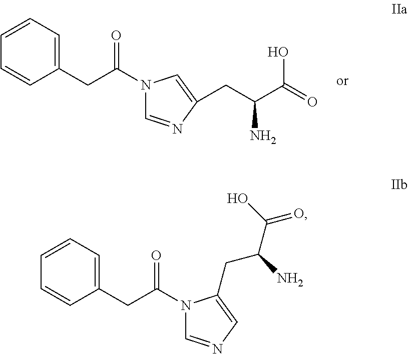
or a pharmaceutically acceptably salt or a prodrug thereof.

18-21. (canceled)

22. A method of preparing a compound of Formula Ia or Ib: 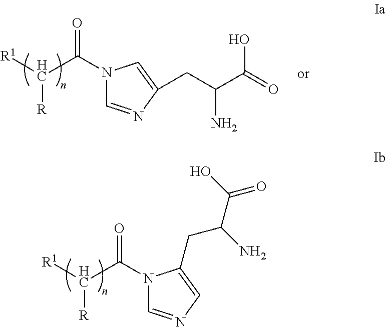
wherein:
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
or a stereoisomer thereof,
comprising treating a compound of formula III: 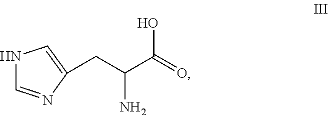
or a stereoisomer thereof,
with an acyl halide of formula IV: 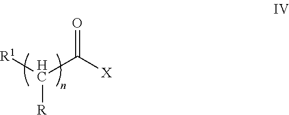
wherein
X is halo;
n is 0, 1, 2, or 3;
R is independently selected from the group consisting of hydrogen, halo, and alkyl; and
R¹ is selected from the group consisting of aryl, substituted aryl, heteroaryl and substituted heteroaryl,
under suitable reaction conditions to give the compound of formula Ia or Ib, respectively.

23. The method of claim 22, wherein X is chloro, fluoro, or bromo; n is 1; R is hydrogen or methyl; and R¹ is aryl or substituted aryl.

24. (canceled)

25. The method of claim 22, wherein X is chloro; n is 1; R is hydrogen; and R¹ is aryl.

26. A method for activating apoptosis in a cell, comprising contacting the cell with an effective amount of a compound of claim 1.

27. (canceled)

28. The method of claim 26, wherein the cell is a tumor cell.

29. The method of claim 28, wherein the tumor cell is one or more of the group an adenocarcinoma, a leukemia, a lymphoma, a melanoma, a myeloma, a sarcoma or a teratocarcinoma cell.

30. The method of claim 28, wherein the tumor cell is a tumor cell of one or more of the group adrenal gland, bladder, bone, bone marrow, brain, breast, cervix, gall bladder, ganglia, gastrointestinal tract, heart, kidney, liver, lung, muscle, ovary, pancreas, parathyroid, penis, prostate, salivary glands, skin, spleen, testis, thymus, thyroid or uterus.

31. The method of claim 28, wherein the tumor cell is a brain tumor cell or a gastrointestinal tumor cell.

32. The method of claim 26, wherein the contacting is in vitro or in vivo.

33-49. (canceled)
